(19) United States
(12) Patent Application Publication
(10) Pub. No.: US 2010/0173354 A1
(43) Pub. Date: Jul. 8, 2010

(54) METHOD FOR THE FERMENTATION OF ENSILAGED RENEWABLE RAW MATERIALS

(75) Inventors: Bjoern Schwarz, Dresden (DE); Burghardt Fassauer, Dresden (DE); Hannelore Friedrich, Radebeul (DE); Eberhard Friedrich, Radebeul (DE); Alexander Michaelis, Dresden (DE)

Correspondence Address: GREENBLUM & BERNSTEIN, P.L.C., 1950 ROLAND CLARKE PLACE, RESTON, VA, 20191, US

(73) Assignee: Fraunhofer-Gesellschaft Zur Foerderung Der Angewandten Forschung E.V., Muenchen (DE)

(21) Appl. No.: 12/593,192

(22) PCT Filed: Mar. 20, 2008
(86) PCT No.: PCT/EP08/53425

§ 371 Date: Jan. 19, 2010

(30) Foreign Application Priority Data
Mar. 27, 2007 (DE) 10 2007 017 358.1
Oct. 8, 2007 (DE) 10 2007 000 834.3

(51) Int. Cl. C12P 1/00

(52) U.S. Cl. 435/41

(57) ABSTRACT

A method of fermenting ensilaged renewable raw materials, wherein the method comprises washing and crushing ensilaged renewable raw materials, removing at least some water from the washed and crushed ensilaged renewable raw materials, subjecting the washed and crushed ensilaged renewable raw materials to hydrolysis, and subjecting hydrolysis products to a biogas production method in fermenters

### CROSS-REFERENCE TO RELATED APPLICATIONS

The present application is a U.S. National Stage of International Patent Application No. PCT/EP2008/053425 filed Mar. 20, 2008 which published as WO 2008/116842 on Oct. 2, 2008, and claims priority of German Patent Application Nos. 10 2007 017 358.1 filed Mar. 27, 2007 and 10 2007 000 834.1 filed Oct. 8, 2007.

### BACKGROUND OF THE INVENTION

1. Field of the Invention

The invention relates to the fields of biochemistry and energy production and relates to a method for the fermentation of ensilaged renewable raw materials, which, subsequently used in a biogas production facility, exhibit improved properties. A use is possible in the monofermentation of renewable raw materials as well as in the co-fermentation with commercial fertilizers (e.g., liquid manure) in agricultural biogas facilities or in the co-fermentation with sewage sludge in municipal sewage treatment plants.

2. Discussion of Background Information

The conversion of biomass into biogas to be energetically recovered while utilizing the biochemical capacity of an anaerobic mixed population of microorganisms is practiced on an industrial scale in agricultural biogas facilities as well as in the digestion towers of municipal sewage treatment plants. The process engineering used thereby covers a very broad spectrum of combinations and number and switching of fermenters, process temperature (mesophilic, thermophilic), substrate treatment, charging regime, intermixing, retention time and organic load.

In the utilization of renewable raw materials as the main substrate or co-substrate for biogas production, the chemical structure thereof prevents a complete conversion into biogas. Large proportions of this plant material are composed of cellulose, hemicellulose and lignin hardly accessible or not accessible at all for microorganisms. Moreover, the particle size of the ensilaged raw materials lies in the centimeter range and is therefore relatively coarse. Approximately 60-80% of the dry matter has a particle size of more than 1 mm. The ratio of circumference/area as a measure of the specific surface area of this coarse fraction is on average 1-2 mm/mm². This specific surface area per substrate quantity which hydrolytically acting microorganisms and enzymes can attack for the transformation of matter is comparatively small. The particle size as well as the chemical structure lead to unsatisfactory and in part uneconomic degradation ratios with the application of conventional fermentation technologies. At 50 to 150 days, the dwell times of the substrates in anaerobic fermenters according to the prior art are very long and the degradation ratios achieved are at the same time unsatisfactory, which has a negative effect on the cost-effectiveness of the facilities.

The various charge substrates are either mixed (mashed) with one another in a preliminary tank or fed separately into the fermenter. A targeted biological prehydrolysis or crushing is rarely practiced. However, as is known, hydrolysis represents the step in the anaerobic degradation chain that limits the speed. For this reason the realization thereof in the actual fermenter together with all of the other degradation steps is to be rated as crucial. In the fermenter the environment conditions are established as the result of all of the biochemical processes taking place. These conditions are not to be evaluated as optimal in particular for hydrolysis, so that a decoupling of this step with the establishment of the best possible conditions should be state of the art, but is not for ensilaged materials.

The problem with the prehydrolysis of ensilaged materials is their very high content of organic acids, which during the ensilagation process are produced as natural preservatives. The pH value of a hydrolysis stage operated with silage without corresponding buffer substances falls into a range that does not permit any further release of organic acids (preservation/self-inhibition). In the co-fermentation of silage with liquid manure, although the buffer effect of the liquid manure is sufficient to create environment conditions for a biological hydrolysis, the process of the desired substrate solution is limited by the load of organic acids in the silage (rapid gradient adjustment). This means that a stage of this type does not work efficiently enough based on the easily accessible constituents released within a time unit.

In the course of the expansion of the generation of renewable energy, the use of renewable (ensilaged) raw materials has gained considerable importance. Since in contrast thereto the quantity of liquid manure available is to be considered constant, at present and in the future an increasing number of facilities will be installed which omit liquid manure largely or completely. The use of an upstream hydrolysis stage is rendered much more difficult for facilities of this type, since a buffer substrate for neutralizing the silage acids suitable for liquid manure has not been available so far.

Furthermore, with reactors switched in series (cascades) only the first reactor is utilized to full capacity, since the largest proportion of the microbiologically available organic substances are already converted in the first 20 to 30 days. All of the downstream reactors are very limited in their degradation activity and speed. The reason for this is the very slow hydrolysis of the remaining organic fractions. This leads to an under-utilization of the methanogenesis, which still has marked reserves.

In the energy recovery of the biogas formed, the quality thereof for the systems used is very important. The content of hydrogen sulfide and methane should be particularly emphasized here. While the former has an impact on the operating stability due to corrosion, a higher methane content means a greater power density and thus, for example, a higher efficiency of a combined heat and power plant. According to the prior art, the methane content of biogas facilities is not directly influenced, but as a rule is dependent on the substrate used. The exception is the processing for feeding to gas or fuel networks for which a multiplicity of technical solutions are available, which are expensive to operate in terms of energy. Biological desulfurization (O₂ charge) as well as external desulfurization plants are used for the reduction of the hydrogen sulfide content.

In an open hydrolysis stage in particular carbon dioxide and hydrogen sulfide are emitted into the atmosphere. These separated reaction products are missing in the biogas of the subsequent fermentation stage, which is why the quality thereof improves.

The disadvantages of the known technical solutions lie in the comparatively long reaction time and the in part substantial fluctuations in quality of the properties of the biogas produced.

### SUMMARY OF THE INVENTION

The invention relates to a method for fermenting ensilaged renewable raw materials, through which the total times for the production of biogas are reduced, and wherein the methane yields are increased and a lower variation range in the quality of the biogas produced is achieved.

In the method according to the invention for fermenting ensilaged renewable raw materials, ensilaged renewable raw materials are washed and crushed, thereafter the washed and crushed ensilaged renewable raw materials, from which at least a part of the washing water has been removed, are subjected to a separate hydrolysis, and subsequently the hydrolysis products are subjected to the known method for biogas production in fermenters.

Advantageously, the ensilaged renewable raw materials are mixed or sprayed with the washing water.

Furthermore advantageously, low-viscosity substances that do not have any disadvantageous effects on the subsequent anaerobic degradation steps in the method for biogas production in fermenters, are used as washing water, wherein particularly advantageously liquid waste, industrial water, drinking water or process water from dehydration steps are used as washing water.

Likewise advantageously a quantity of 20 to 500% by weight washing water based on the silage mass (original substance) to be washed is used.

It is furthermore advantageous if the washing of the ensilaged renewable raw materials is carried out with targeted intermixing of the raw materials.

It is also advantageous if the washing of the ensilaged renewable raw materials is carried out at temperatures in the range of 1° C. to 60° C.

It is also advantageous if the washing of the ensilaged renewable raw materials is carried out in a period from 1 s to 10 h.

It is advantageous if the washing water is removed from the washed silage by way of pressing, filtering or separation in the gravitational field or centrifugal force field.

It is furthermore advantageous if the ensilaged raw materials are mechanically crushed before the washing.

It is likewise advantageous if the ensilaged raw materials mixed with washing water are mechanically crushed simultaneously during the washing and dewatering process.

It is also advantageous if the ensilaged and at least partially dewatered renewable raw materials are mechanically crushed.

It is also advantageous if the mechanical crushing is carried out by way of cutting, squeezing, rubbing and shredding.

It is also advantageous if the mechanical crushing is carried out within 1 s-10 min.

It is likewise advantageous if 10%-40% liquid manure or 10%-70% digestate from the facility's biogas extraction process or 5%-25% liquid manure together with 5-25% digestate is added to the hydrolysis process in addition to the washed ensilaged and at least partly dewatered renewable raw materials, based on the total mixture produced, wherein all of the variants can be combined with 0%-50% activated sludge from municipal sewage treatment plants and/or 0%-50% process water.

It is also advantageous if the at least partially removed washing water is metered in the fermenters in the following process steps for biogas production.

With the method according to the invention it is possible to accelerate the entire process for producing biogas from ensilaged renewable raw materials and to achieve the desired shortening of the process times as a whole.

At the same time, the methane quantity produced per substrate quantity used is increased and the quality of the properties of the biogas produced is improved.

Furthermore, with the method according to the invention the prerequisite is created for the operation of a biological hydrolysis stage for the acidification of ensilaged substrates without the mandatory use of a larger quantity of liquid manure. It is thus possible to place at the start a process step uncoupled from the actual fermentation stage for the production of biogas, which under optimal environment conditions accelerates the step of hydrolysis that limits the speed. The dwell time necessary in the subsequent fermentation step is shortened, whereby the reactor sizes and thus the necessary investment costs are reduced.

In the use of fermenters connected in series, the individual process steps are more uniformly loaded and the overload of the first fermenter is transferred in part to the following fermenters. The entire process is stabilized and the gas yield increased for each substrate load supplied.

The gas quality is improved with respect to the methane and hydrogen sulfide content.

This is achieved in that the described self-inhibition of the hydrolysis through organic acids introduced from the silages is eliminated or reduced through the washing of the ensilaged renewable raw materials. Furthermore, the mixing behavior of the raw materials and the reactivity thereof are markedly improved through the strongest possible mechanical crushing of the ensilaged renewable raw materials before, during or after the washing. This is achieved in particular through the enlargement of the surface of the raw materials. The hydrolysis process is further accelerated through this process stage of mechanical crushing according to the invention. The return of digestates into the hydrolysis stage is very important to buffer the pH value and for the supply of hydrolyzing microorganisms.

First of all, the ensilaged renewable raw materials are washed, advantageously this is carried out through the mixing or spraying of the silage to be used with washing water, wherein the washing water is used in a quantity between 20% by weight and 500% by weight based on the silage mass to be washed (damp mass−original silage). Low-viscosity (0-5% dry matter contents) substances which are available and do not have any harmful effect on a subsequent anaerobic degradation step for producing biogas can be used as a washing medium. Advantageously, liquid waste, industrial water, drinking water or filtrates from dewatering stages are used to this end.

The contact time between washing water and silage is advantageously 1 s to 10 h. Likewise it is advantageous to carry out an active intermixing during the contact period through a mechanical movement of the silage with the washing water.

Thereafter at least a partial separation of the washing water from the silage is necessary. Advantageously, at least 50% of the washing water should be removed. A large part can already thereby be removed with the aid of gravitational force or centrifugal force or by pressing. However, a support of this process through the use of mechanical units is preferable (e.g., screw separator). A very high quantity of press water of 100-200% compared to the washing water quantity originally used can thus also advantageously be achieved.

Two products are obtained as a result of the washing stage according to the invention. On the one hand a removed washing water is produced, which is as free as possible of coarse particles and heavily loaded with organic acids and other dissolved, easily degradable substrates and advantageously can be fed to the fermenters as a rapidly recyclable substrate. One particular advantage is the very easy handling which renders possible a uniform metering. In the case of single-stage plants, a metering in charging intervals for the advantageous homogenization of the charging load is possible. In the case of multiple-stage plants, the addition of the separated washing water is advantageous in particular in the secondary or further fermenters. The latter leads to a relief of the load on the first fermenter, which is generally heavily loaded anyway, and to a better utilization of existing capacities.

The washed and at least partially dewatered silage, which in terms of its properties (dry residue, handling) is very similar to the unwashed silage, is obtained as a second product. However, the crucial difference is the load of dissolved substances, such as, e.g., the organic acids, which is now reduced by 20% to 80%.

The mechanical crushing of the ensilaged raw materials can be carried out according to the invention before (raw silage) as well as after (compacted material) the washing. A major advantage is also provided by the third possibility of incorporating a crushing in which the silage is simultaneously mechanically crushed during the washing process, for example, while the washing water is pressed out. The latter reduces the expenditure in terms of machinery, since only one unit is required for washing and crushing.

The mechanical crushing of the (washed) silage advantageously takes place in cutting mills, extruders or impact mills, wherein a cutting, squeezing, rubbing and shredding of the coarse constituents is carried out. The loading time is between 1 s and 10 min. After the treatment, the proportion of particles >1 mm is only 20%. Moreover, for this coarse content a ratio of circumference/area of the particles of approx. 6-10 mm/mm² is achieved.

The washed and crushed compacted material subsequently reaches the hydrolysis stage. In this stage, based on the total mixture produced, a mixing with 10%-70% digestate, which is returned from the downstream fermentation, and 0%-50% activated sludge from municipal sewage treatment plants and/or 0% to 50% process water is possible. A further possibility is the mixing with 10%-40% liquid manure and 0%-50% activated sludge from municipal sewage treatment plants and/or 0% to 50% process water. An addition of 5-25% digestate and 5-25% liquid manure combined with the referenced portions of activated sludge and process water is also a possible variant. Through the mashing with the referenced substrates the silage is converted into a stirrable state (dry residue=7-15%), the pH value buffered and a sufficient quantity of active microorganisms fed to the process stage. A mechanical crushing of the material provides further advantages for this. The return of digestate or dewatered digestate (liquid portion) to the hydrolysis stage is particularly advantageous with the omission of the use of liquid manure. The solids of the silage used are converted into solution in part with a dwell time of 6 h to 5 days (depending on the agitation intensity and process temperature) in the hydrolysis stage. The substances released are easily available in the subsequent fermentation stage and lead to an accelerated gas formation.

In the case of a facility with two fermenters, according to the method according to the invention a dwell time of 20-30 days is set in the first fermenter. For the subsequent fermenter 10-20 days are then sufficient, since it receives on the one hand the outflow from the main fermenter with lower gas potential and on the other hand the press water from the washing stage with very quick conversion times as input. The total dwell time in the fermenters is thus advantageously reduced.

Compared to solutions of the prior art, an acceleration of the anaerobic degradation of ensilaged renewable raw materials occurs as well as an increase in the methane yield per substrate used. The use of liquid manure for the operation of the hydrolysis stage can be omitted, which makes the site of the biogas facility independent of the presence of liquid manure or livestock operations. This aspect is of particular interest when it is a matter of a combination of waste disposal plants and renewable raw materials.

Furthermore, the gas quality, the process stability and the utilization of the existing capacities are improved. The latter is due in particular to the flexibility in the use of the press water produced.

A washing and crushing of the ensilaged charge substrates with subsequent hydrolysis also provides the cited advantages for existing plants that operate with liquid manure.

The invention also provides for a method of fermenting ensilaged renewable raw materials, wherein the method comprises washing and crushing ensilaged renewable raw materials, removing at least some water from the washed and crushed ensilaged renewable raw materials, subjecting the washed and crushed ensilaged renewable raw materials to hydrolysis, and subjecting hydrolysis products to a biogas production method in fermenters.

The hydrolysis may be a separate hydrolysis and the biogas production is a conventional biogas production method. The method may further comprise one of mixing the ensilaged renewable raw materials with washing water and spraying the ensilaged renewable raw materials with washing water. The washing water used in the washing may comprise low-viscosity substances that do not have any disadvantageous effects on subsequent anaerobic degradation during the biogas production method. The washing water used in the washing comprises may be one of liquid waste, industrial water, drinking water, process water from dehydration.

The washing water used in the washing may comprise a quantity of 20 to 500% by weight based on a silage mass (original substance). The washing may utilize a targeted intermixing of the ensilaged renewable raw materials. The washing may be carried out at temperatures in the range of 1° C. to 60° C. The washing may be carried out for a period of between 1 second and 10 hours.

The removing may comprise removing washing water utilizing one of pressing, filtering, gravity separation, and centrifugal separation. The washing and crushing may comprise before the washing, mechanically crushing the ensilaged renewable raw materials. The method may further comprise, before the washing and crushing, mixing the ensilaged renewable raw materials with washing water. The washing and crushing may comprise simultaneously washing and mechanically crushing the ensilaged renewable raw materials. The washing and crushing may comprise simultaneously washing, mechanically crushing, and dewatering the ensilaged renewable raw materials.

The crushing may comprise mechanically crushing the ensilaged renewable raw materials and at least partially dewatered renewable raw materials. The crushing may comprise cutting, squeezing, rubbing, and shredding. The crushing may be carried out for a period of between 1 second and 10 minutes.

The method may further comprise adding to the hydrolysis one of 10%-40% liquid manure, 10%-70% digestate from the biogas production method, and 5%-25% liquid manure together with 5-25% digestate.

The method may further comprise adding to the hydrolysis washed ensilaged renewable raw materials and at least partly dewatered renewable raw materials. The method may further comprise adding to the hydrolysis at least one of 0%-50% activated sludge from municipal sewage treatment plants and 0%-50% process water.

The method may further comprise metering in the fermenters the removed water.

The invention also provides for a method of producing a biogas comprising washing a silage comprising renewable raw materials, removing washing water from the washed and crushed silage, crushing the silage, subjecting the washed and crushed silage to hydrolysis, and subjecting products of the hydrolysis to fermentation.

The invention also provides for a method of producing a biogas from a silage comprising renewable raw materials, wherein the method comprises washing the silage using service water, removing some of the service water from the washed and crushed silage, crushing the silage, subjecting the washed and crushed silage to hydrolysis, subjecting products of the hydrolysis to fermentation, and producing a biogas.

### BRIEF DESCRIPTION OF THE DRAWINGS

The invention is described in more detail below based on two exemplary embodiments wherein:

FIG. 1 shows a diagram of the total process for the production of biogas with the hydrolysis process stage; and

FIG. 2 shows a diagram of the total process for the production of biogas with the crushing and hydrolysis stage.

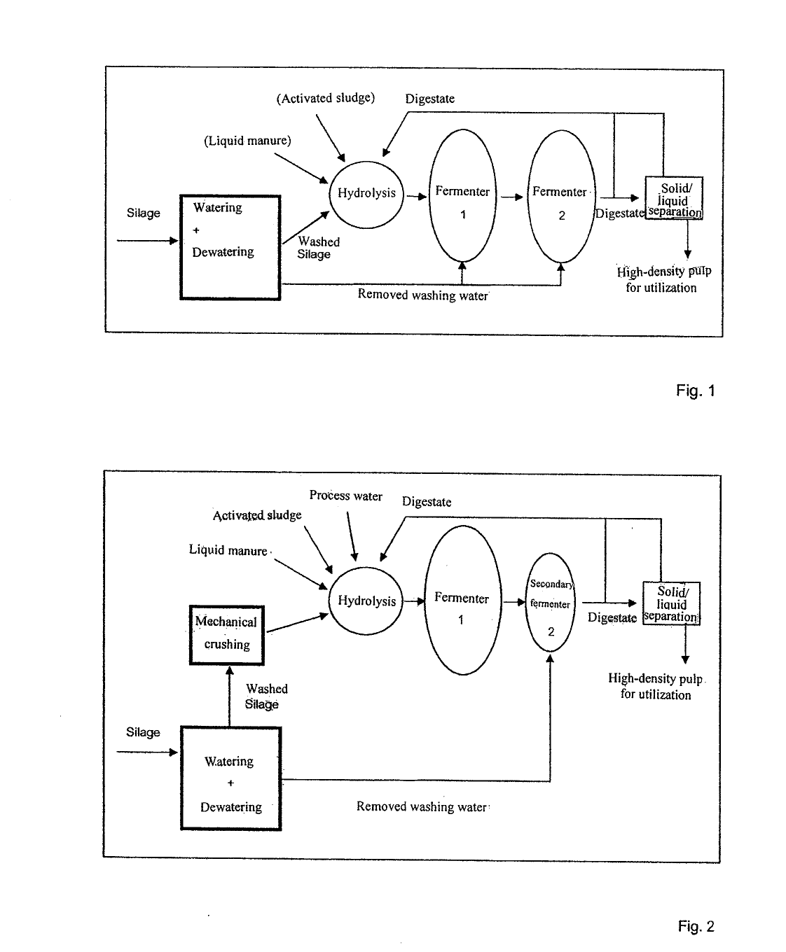

### DETAILED DESCRIPTION OF THE INVENTION

### Example 1

In this example, 1000 kg silage, comprising 60% corn and 40% rye whole crop silage is fed to a washing reactor. Subsequently 1000 liters (l) of liquid, which comprises service water (sewage treatment plant outflow), is added to the washing reactor. After the liquid is poured in, the silage is moved for 10 min by mixing plungers. Thereafter the washed silage remains in the washing reactor for 5 min, wherein 100% of the washing water is removed from the silage through the compression of the silage. The washing water that is pressed out is collected. It has a composition of 2.5% dry content and 50 grams/liter (g/1) dissolved CSB and is added to the existing fermenters in the following process steps. The washed and partially dewatered silage is fed to a hydrolysis reactor to which 0% by weight liquid manure, 15% by weight activated sludge from a municipal sewage treatment plant and 50% by weight of digestate from the facility's biogas production process is added. The substances remain in the hydrolysis reactor for 2 days and are then fed to the known method for biogas production.

The entire process for biogas production requires a period of 37 days according to the invention, compared to 60 days according to methods according to the prior art. Furthermore, a standardization of the composition occurs through the washing of the silage, so that the hydrolyzed silage fed to the known biogas production method has a more homogeneous composition, whereby the biogas produced likewise has an improved gas quality.

### Example 2

In this example, 1000 kg silage, comprising 60% corn and 40% rye whole crop silage is fed to a washing reactor. Subsequently, 500 l of liquid, which comprises service water (sewage treatment plant outflow) is added to the washing reactor. Thereafter the washed silage remains in the washing reactor for 5 min, whereby the washing water seeps through the silage body due to the force of gravity and collects on the bottom. By emptying the entire container, the water and the silage are intermixed again, and a further mechanical mixing is not carried out. This silage/water mixture is fed to an extruder by way of a conveyor device and the washing water is pressed out there. As a result of the dewatering, approx. 800 l of press water with 4.5% dry matter content and 55 g/l dissolved CSB is obtained. This press water is fed completely to the subsequent fermenter of the two-stage device switched in series. The washed and partially dewatered silage is continuously crushed with the aid of a planetary gear extruder, wherein the coarse substances >1 mm are reduced from a mass portion of 80% to 20%, or 75% of these coarse substances are crushed to below 1 mm. The dwell time in the unit is approx. 15 seconds (s), wherein the ratio of circumference to area of the particles increases from 1.5 to 9 mm/mm².

Subsequently, the washed, pressed and crushed silage is fed to a hydrolysis reactor, to which are fed 0% by weight liquid manure, 10% by weight activated sludge of a municipal sewage treatment plant, and 65% by weight digestate from the facility's biogas production method. The substances remain in the hydrolysis reactor for 2 days and are then fed to the first fermentation step in the first fermenter, in which the hydraulic dwell time is 25 days. Subsequently, the products are guided into the secondary fermenter and remain there on average for another 10 days.

The entire method for biogas production requires a period of 37 days according to the invention, compared to 60 days according to methods according to the prior art. Furthermore, a homogenization of the composition is achieved through the washing and mechanical crushing of the silage, so that the hydrolyzed silage fed to the known biogas production method has a more uniform composition, whereby the biogas produced likewise has an improved gas quality.

1-16. (canceled)

17. A method of fermenting ensilaged renewable raw materials, the method comprising:
washing and crushing ensilaged renewable raw materials;
removing at least some water from the washed and crushed ensilaged renewable raw materials;
subjecting the washed and crushed ensilaged renewable raw materials to hydrolysis; and
subjecting hydrolysis products to a biogas production method in fermenters.

18. The method of claim 17, wherein the hydrolysis is a separate hydrolysis and the biogas production is a conventional biogas production method.

19. The method of claim 17, further comprising one of mixing the ensilaged renewable raw materials with washing water; and
spraying the ensilaged renewable raw materials with washing water.

20. The method of claim 17, wherein washing water used in the washing comprises low-viscosity substances that do not have any disadvantageous effects on subsequent anaerobic degradation during the biogas production method.

21. The method of claim 17, wherein washing water used in the washing comprises one of:
liquid waste;
industrial water;
drinking water;
process water from dehydration.

22. The method of claim 17, wherein washing water used in the washing comprises a quantity of 20 to 500% by weight based on a silage mass (original substance).

23. The method of claim 17, wherein the washing utilizes a targeted intermixing of the ensilaged renewable raw materials.

24. The method of claim 17, wherein the washing is carried out at temperatures in the range of 1° C. to 60° C.

25. The method of claim 17, wherein the washing is carried out for a period of between 1 second and 10 hours.

26. The method of claim 17, wherein the removing comprises removing washing water utilizing one of:
pressing;
filtering;
gravity separation; and
centrifugal separation.

27. The method of claim 17, wherein the washing and crushing comprises:
before the washing, mechanically crushing the ensilaged renewable raw materials.

28. The method of claim 17, further comprising, before the washing and crushing:
mixing the ensilaged renewable raw materials with washing water.

29. The method of claim 17, wherein the washing and crushing comprises:
simultaneously washing and mechanically crushing the ensilaged renewable raw materials.

30. The method of claim 17, wherein the washing and crushing comprises:
simultaneously washing, mechanically crushing, and dewatering the ensilaged renewable raw materials.

31. The method of claim 17, wherein the crushing comprises:
mechanically crushing the ensilaged renewable raw materials and at least partially dewatered renewable raw materials.

32. The method of claim 17, wherein the crushing comprises cutting, squeezing, rubbing, and shredding.

33. The method of claim 17, wherein the crushing is carried out for a period of between 1 second and 10 minutes.

34. The method of claim 17, further comprising adding to the hydrolysis one of:
10%-40% liquid manure;
10%-70% digestate from the biogas production method; and
5%-25% liquid manure together with 5-25% digestate.

35. The method of claim 34, further comprising adding to the hydrolysis washed ensilaged renewable raw materials and at least partly dewatered renewable raw materials.

36. The method of claim 35, further comprising adding to the hydrolysis at least one of:
0%-50% activated sludge from municipal sewage treatment plants; and
0%-50% process water.

37. The method of claim 17, further comprising metering in the fermenters the removed water.

38. A method of producing a biogas comprising:
washing a silage comprising renewable raw materials;
removing washing water from the washed and crushed silage;
crushing the silage;
subjecting the washed and crushed silage to hydrolysis; and
subjecting products of the hydrolysis to fermentation.

39. A method of producing a biogas from a silage comprising renewable raw materials, the method comprising:
washing the silage using service water;
removing some of the service water from the washed and crushed silage;
crushing the silage;
subjecting the washed and crushed silage to hydrolysis;
subjecting products of the hydrolysis to fermentation; and
producing a biogas.
